(19) United States
(12) Patent Application Publication
(10) Pub. No.: US 2025/0139809 A1
(43) Pub. Date: May 1, 2025

(54) METHOD AND APPARATUS FOR DETECTING ISOLATION PAD IN MEDICAL IMAGING AND MEDICAL IMAGING SYSTEM

(71) Applicant: GE Precision Healthcare LLC, Waukesha, WI (US)

(72) Inventors: Qingyu Dai, Beijing (CN); Xiaolan Liu, Beijing (CN); Gang Hu, Beijing (CN); Ting Ye, Beijing (CN); Jun Zhang, Beijing (CN); Jian Cui, Beijing (CN); Kun Wang, Beijing (CN)

(21) Appl. No.: 18/907,900

(22) Filed: Oct. 7, 2024

(30) Foreign Application Priority Data
Oct. 25, 2023 (CN) 202311391624.6

(51) Int. Cl. G06T 7/70; G06T 3/40; G06T 7/00; G06T 7/50; G06T 7/90

(52) U.S. Cl. CPC G06T 7/70; G06T 3/40; G06T 7/0012; G06T 7/50; G06T 7/90; G06T 2207/10024; G06T 2207/10028; G06T 2207/30004

(57) ABSTRACT

A method and an apparatus for detecting an isolation pad in medical imaging, and a medical imaging system are provided. The method includes: acquiring an image of a detection object captured via a camera; determining, based on the image, whether an isolation pad is present at a predetermined part of the detection object; and outputting information related to a result of the determination. The present application detects an isolation pad placed on the body of a detection object, and it is thus possible to reliably avoid the formation of a loop in the body of the detection object.

### CROSS REFERENCE TO RELATED APPLICATIONS

The present application claims priority and benefit of Chinese Patent Application No. 202311391624.6 filed on Oct. 25, 2023, which is incorporated herein by reference in its entirety.

### TECHNICAL FIELD

Embodiments of the present application relate to the technical field of medical devices, and in particular to a method and apparatus for determining an isolation pad in medical imaging, and a medical imaging system.

### BACKGROUND

In a scenario in which a medical imaging system is used to scan and image a detection object, a loop is sometimes formed in the body of a scanned object. The loop is formed by exposed parts of the body of the scanned object coming into contact with each other, for example, upper limbs (e.g., hands) of the scanned object coming into contact with each other, an upper limb of the scanned object coming into contact with a side of the body, or the lower limbs of the scanned object coming into contact with each other (e.g., inner sides of the two thighs coming into contact with each other, or the two feet coming into contact with each other, etc.).

The appearance of a loop sometimes causes adverse effects for the scanned object. For example, when a detection object is scanned and imaged by a magnetic resonance imaging (MRI) system, a loop in the body of the scanned object may cause phenomena such as injury to the scanned object.

In the prior art, a loop generated in the body of a detection object is detected, so as to avoid adverse effects on the detection object.

It should be noted that the above introduction of the background is only for the convenience of clearly and completely describing the technical solutions of the present application, and for the convenience of understanding for those skilled in the art.

### SUMMARY

The inventors of the present application have found that, in the prior art, a posture of a scanned object on a scanning bed is usually detected to determine whether a loop is formed in the body of a detection object. However, this detection method is sometimes inaccurate, and furthermore, a new loop may be generated by movement of a body part during scanning. Therefore, loop formation cannot be reliably avoided by means of detecting the posture of the detection object before scanning.

To solve the above technical problems or at least similar technical problems, embodiments of the present application provide a method and apparatus for detecting an isolation pad in medical imaging, and a medical imaging system. In said method, an isolation pad placed on the body of a detection object is detected, and it is thus possible to reliably avoid the formation of a loop in the body of the detection object.

According to one aspect of the embodiments of the present application, a method for detecting an isolation pad in medical imaging is provided. The method includes: acquiring an image of a detection object captured via a camera; determining, based on the image, whether an isolation pad is present at a predetermined part of the detection object; and outputting information related to a result of the determination.

According to one aspect of the embodiments of the present application, an apparatus for detecting an isolation pad in medical imaging is provided. The apparatus includes: an acquisition unit for acquiring an image of a detection object captured via a camera; and a determination unit for determining, based on the image, whether an isolation pad is present at a predetermined part of the detection object. The apparatus also includes a prompt unit, outputting information related to a result of the determination.

According to one aspect of the embodiments of the present application, a medical imaging system is provided. The system includes: a controller configured to execute the above method for detecting an isolation pad.

One of the beneficial effects of the embodiments of the present application is that the present method avoids determining a loop based on the posture of the scanned object; rather a loop is determined by means of detecting an isolation pad arranged on the body of the detection object. Therefore, the problem of posture determination is converted into the problem of determining the presence or absence of an isolation pad, and determining the presence or absence of an isolation pad is more accurate. Therefore, it is possible to reliably avoid the formation of a loop in the body of the detection object.

With reference to the following description and drawings, specific implementations of the embodiments of the present application are disclosed in detail, and the means by which the principles of the embodiments of the present application can be employed are illustrated. It should be understood that the embodiments of the present application are not limited in scope thereby. Within the scope of the spirit and clauses of the appended claims, the embodiments of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of a magnetic resonance imaging system according to an embodiment of the present application;

FIG. 2 is a schematic diagram of placing an isolation pad at a predetermined part of a detection object;

FIG. 3 is another schematic diagram of placing an isolation pad at a predetermined part of a detection object;

FIG. 4 is a schematic diagram of a method for detecting an isolation pad in medical imaging according to an embodiment of the present application;

FIG. 5 is a schematic diagram of operation 402;

FIG. 6 is a schematic diagram of a predetermined pattern of an isolation pad;

FIG. 7 is a schematic diagram of an image captured by a camera when a detection object is in a first posture;

FIG. 8 is a schematic diagram of an image captured by a camera when a detection object is in a second posture;

FIG. 9 is a schematic flowchart of a specific implementation of a method for detecting an isolation pad in medical imaging according to an embodiment of the present application;

FIG. 10 is a schematic diagram of an apparatus for detecting an isolation pad according to an embodiment of the present application; and

FIG. 11 is a schematic diagram of a medical imaging device according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “based on” should be construed as “at least in part based on . . . ”, unless otherwise specified in the context.

In the embodiments of the present application, the term “key point” may be equivalently replaced with “key coordinate point”, “landmark”, “landmark point”, etc. The term “object” may be equivalently replaced with “detection object”, “object under detection”, “object being scanned”, “object to be scanned”, “patient”, “object of study”, or the like, which may be a human being or an animal, or may be other objects.

In the embodiments of the present application, the term “include/comprise” when used herein refers to the presence of features, integrated components, steps, or assemblies, but does not preclude the presence or addition of one or more other features, integrated components, steps, or assemblies.

The features described and/or illustrated for one implementation may be used in one or more other implementations in the same or similar manner, be combined with features in other embodiments, or replace features in other implementations.

In the embodiments of the present application, a method for determining a posture of an object or an apparatus for determining a posture of an object may be applicable to various medical imaging scenarios, including, but not limited to, magnetic resonance imaging (MRI), computed tomography (CT), ultrasound imaging, positron emission computed tomography (PET), single photon emission computed tomography (SPECT), PET/CT, PET/MR, or any other suitable medical imaging scenarios.

In the embodiments of the present application, the method, apparatus and system of the present application are exemplarily described by taking an MRI scenario as an example. It should be understood that the contents of the embodiments of the present application are also applicable to other medical imaging scenarios.

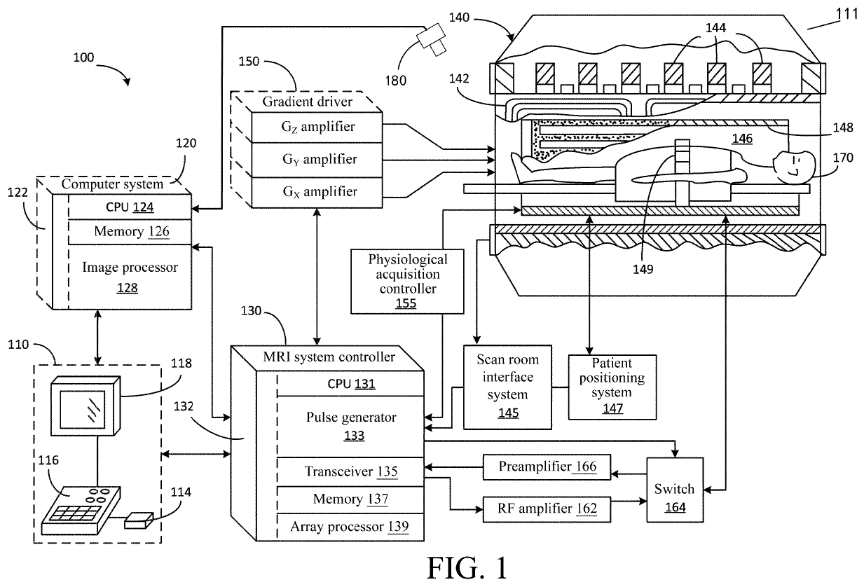

For ease of understanding, FIG. 1 is a schematic diagram of a magnetic resonance imaging (MRI) system 100 according to an embodiment of the present application.

The MRI system 100 includes a scanning unit 111. The scanning unit 111 is used to perform a magnetic resonance scan of a subject (e.g., a human body) 170 to generate image data of a region of interest of the subject 170, wherein the region of interest may be a pre-determined anatomical site or anatomical tissue.

The operation of the MRI system 100 is controlled by an operator workstation 110 that includes an input device 114, a control panel 116, and a display 118. The input apparatus 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and in communication with a computer system 120 that enables an operator to control the generation and display of images on the display 118. The computer system 120 includes various components that communicate with one another via an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced by medical imaging functions implemented in the CPU 124. The computer system 120 may be connected to an archive media device, a persistent or backup memory, or a network. The computer system 120 may be coupled to and communicates with a separate MRI system controller 130.

The MRI system controller 130 includes a set of components that communicate with one another via an electrical and/or data connection module 132. The connection module 132 may employ a direct wired connection, a fiber optic connection, a wireless communication link, etc. The MRI system controller 130 may include a CPU 131, a sequence pulse generator (also known as a pulse generator) 133 in communication with the operator workstation 110, a transceiver (also known as an RF transceiver) 135, a memory 137, and an array processor 139.

In some embodiments, the sequence pulse generator 133 may be integrated into a resonance assembly 140 of the scanning unit 111 of the MRI system 100. The MRI system controller 130 may receive a command from the operator workstation 110, and is coupled to the scanning unit 111 to indicate an MRI scanning sequence to be performed during an MRI scan, so as to be used to control the scanning unit 111 to perform the flow of the aforementioned magnetic resonance scan. The MRI system controller 130 is further coupled to a gradient driver system (also known as gradient driver) 150 and is in communication therewith, and the gradient driver system is coupled to a gradient coil assembly 142 to generate a magnetic field gradient during an MRI scan.

The sequence pulse generator 133 may further receive data from a physiological acquisition controller 155 that receives signals from a plurality of different sensors (e.g., electrocardiogram (ECG) signals from electrodes attached to a patient, etc.), the sensors being connected to a subject or patient 170 undergoing an MRI scan. The sequence pulse generator 133 is coupled to and in communication with a scan room interface system 145 that receives signals from various sensors associated with the state of the resonance assembly 140. The scan room interface system 145 is further coupled to and in communication with a patient positioning system 147 that sends and receives signals to control movement of a patient table to a desired position to perform the MRI scan.

The MRI system controller 130 provides gradient waveforms to the gradient driver system 150, and the gradient driver system includes Gₓ (x direction), G_{y} (y direction), and G_{z} (z direction) amplifiers, etc. Each of the Gₓ, G_{y}, and G_{z} gradient amplifiers excites a corresponding gradient coil in the gradient coil assembly 142, so as to generate a magnetic field gradient used to spatially encode an MR signal during an MRI scan. The gradient coil assembly 142 is disposed within the resonance assembly 140, and the resonance assembly further includes a superconducting magnet having a superconducting coil 144 that, in operation, provides a static uniform longitudinal magnetic field B₀ throughout a cylindrical imaging volume 146. The resonance assembly 140 further includes an RF body coil 148, which, in operation, provides a transverse magnetic field B₁, the transverse magnetic field B₁ being substantially perpendicular to B₀ throughout the entire cylindrical imaging volume 146. The resonance assembly 140 may further include an RF surface coil 149 for imaging different anatomical structures of the patient undergoing the MRI scan. The RF body coil 148 and the RF surface coil 149 may be configured to operate in a transmit and receive mode, a transmit mode, or a receive mode.

The x direction may also be referred to as a frequency encoding direction or a kₓ direction in the k-space, the y direction may be referred to as a phase encoding direction or a k_{y} direction in the k-space, and the z direction may be referred to as a layer surface selection (layer selection) direction. Gₓ can be used for frequency encoding or signal readout, and is generally referred to as a frequency encoding gradient or a readout gradient. G_{y} can be used for phase encoding, and is generally referred to as a phase encoding gradient. G_{z} can be used for slice (layer) position selection to acquire k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses amplified by an RF amplifier 162, and provides the same to the RF body coil 148 through a transmit/receive switch (also known as T/R switch or switch) 164.

As described above, the RF body coil 148 and the RF surface coil 149 may be used to transmit RF excitation pulses and/or receive resulting MR signals from the patient undergoing the MRI scan. The MR signals emitted by excited nuclei in the patient of the MRI scan may be sensed and received by the RF body coil 148 or the RF surface coil 149 and sent back to a preamplifier 166 through the T/R switch 164. The T/R switch 164 may be controlled by a signal from the sequence pulse generator 133 to electrically connect the RF amplifier 162 to the RF body coil 148 in the transmit mode and to connect the preamplifier 166 to the RF body coil 148 in the receive mode. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are stored in the memory 137 for post-processing as a raw k-space data array. A reconstructed magnetic resonance image may be obtained by transforming/processing the stored raw k-space data.

In some embodiments, the MR signals sensed and received by the RF body coil 148 or the RF surface coil 149 and amplified by the preamplifier 166 are demodulated, filtered, and digitized in a receiving portion of the transceiver 135, and transmitted to the memory 137 in the MRI system controller 130. For each image to be reconstructed, the data is rearranged into separate k-space data arrays, each of these separate k-space data arrays is input into the array processor 139, and the array processor is operated to transform the data into an array of image data by Fourier transform.

The array processor 139 uses transform methods, most commonly Fourier transform, to create images from the received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or collectively include a computer processor and a storage medium. The storage medium records a predetermined data processing program to be executed by the computer processor. For example, the storage medium may store a program used to implement scanning processing (such as a scan flow and an imaging sequence), image reconstruction, medical imaging, etc. For example, the storage medium may store a computer program for determining a posture of an object according to the embodiments of the present invention. The described storage medium may include, for example, a ROM, a floppy disk, a hard disk, an optical disk, a magneto-optical disk, a CD-ROM, or a non-volatile memory card.

The inventors have found that, in a method for detecting a posture of a scanned object to determine whether a loop is formed in the body of a detection object, sometimes the loop may not be accurately detected, and misdetection or erroneous detection may easily occur; in addition, a new loop may be generated by the movement of a body part during scanning. Therefore, the formation of the loop cannot be reliably avoided by detecting the posture of the detection object before scanning.

To solve at least one of the above problems, embodiments of the present application provide a method and apparatus for determining an isolation pad in medical imaging, and a medical imaging system.

The method and apparatus for determining an isolation pad in medical imaging, and the medical imaging system, of the present application are based on the premise that an insulating isolation pad having a predetermined thickness (for example, the thickness of the isolation pad being greater than 0.6 cm) may be placed at a predetermined part of a detection object to avoid the formation of a loop in the body of the detection object. For example, before the detection object is imaged and scanned, the isolation pad may be placed between the upper limbs, between an upper limb and a side of the trunk, between two legs, between two feet, etc. of the detection object, so that even if a body part of the detection object moves slightly, exposed parts can be effectively isolated, thereby avoiding the formation of a loop in the body of the detection object.

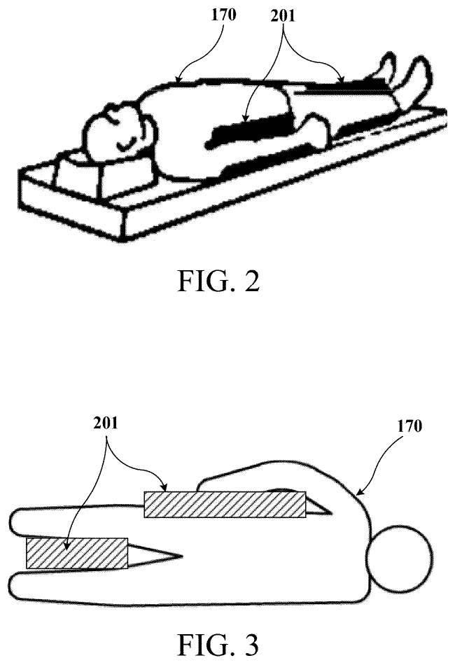

FIG. 2 is a schematic diagram of placing an isolation pad at a predetermined part of a detection object. As shown in FIG. 2, a detection object 170 is in a supine posture, an isolation pad 201 is placed between an arm and a side of the trunk of the detection object 170, an isolation pad 201 is placed between two legs of the detection object 170, etc. In addition, in embodiments of the present application, descriptions related to a supine posture are applicable to a prone posture.

FIG. 3 is another schematic diagram of placing an isolation pad at a predetermined part of a detection object. As shown in FIG. 3, a detection object 170 is in a decub posture, an isolation pad 201 is placed between an arm and a side of the trunk of the detection object 170, an isolation pad 201 is placed between two legs of the detection object 170, etc.

In the embodiments of the present application, based on the above premise, the isolation pad placed on the body of the detection object is detected, and therefore, it is possible to reliably avoid the formation of a loop in the body of the detection object.

Description is made below in conjunction with the embodiments.

Embodiments of the present application provide a method for detecting an isolation pad in medical imaging, which is used to detect, for example, the isolation pad 201 shown in FIG. 2.

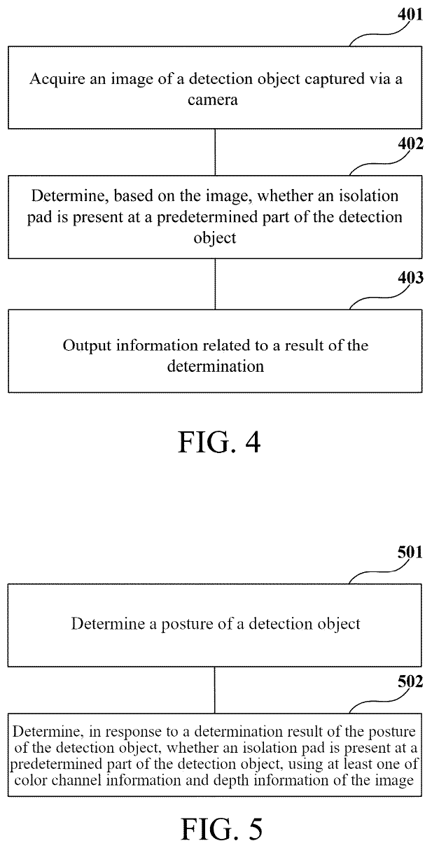

FIG. 4 is a schematic diagram of a method for detecting an isolation pad in medical imaging according to an embodiment of the present application. As shown in FIG. 4, the method includes: at step 401: acquiring an image of a detection object captured via a camera; and determining, based on the image, whether an isolation pad is present at a predetermined part of the detection object at step 402. The method also includes at step 403: outputting information related to a result of the determination.

According to the above embodiment, the isolation pad placed on the body of the detection object is detected. Since a detection result of the isolation pad is more convenient and accurate, the above embodiment can reliably avoid the formation of a loop in the body of the detection object.

In some embodiments, the image in operation 401 may come from a camera 180 shown in FIG. 1. Each frame of the image captured by the camera 180 may include at least one of color channel information and depth information. The color channel information may be stored in the form of a color image, and the depth information may be stored in the form of a depth image.

Each color image may include a plurality of pixel points, and a value of each pixel point may include a value of each color component. The color components may include a red (R) component, a green (G) component, a blue (B) component, and the like.

Each depth image may include a plurality of pixel points, and a value of each pixel point may include a depth value of the pixel. The depth value of the pixel may represent a distance to the camera from a point on a subject (e.g., a shooting object within a shooting range of the camera 180) that corresponds to the pixel.

In some embodiments, the camera 180 may acquire images within a period of time to form an image sequence. In the image sequence, color channel information of the images may be stored separately to form a color image sequence, and depth information of the images may be stored separately to form a depth image sequence. The color image sequence may include a plurality of color images, and the depth image sequence may include a plurality of depth images. Each color image corresponds to a different time instance, and each depth image corresponds to a different time instance. The color images in the color image sequence may be in a one-to-one correspondence to the depth images in the depth image sequence. For example, the color images in the color image sequence may be aligned in time with the depth images in the depth image sequence. The present application is not limited thereto. The color images in the color image sequence may also be in other correspondence relationships with the depth images in the depth image sequence.

In some embodiments, the camera may be placed near (e.g., on an upper side of) the scanning unit 111 shown in FIG. 1, to capture the detection object 170 from an overhead perspective and obtain an image. For example, it may be placed at the position of the camera 180 as shown in FIG. 1. In addition, the present application is not limited thereto, and the camera may also be disposed at other positions.

In some embodiments, different postures of the detection object in a scanning space may result in different shapes or sizes of the isolation pad 201 in the image. Therefore, in operation 402, the isolation pad is detected using a method corresponding to the posture of the detection object, so the isolation pad can be more accurately detected.

The posture of the detection object may include, for example, which direction the face of the detection object is facing, that is, an orientation of the detection object; for example, the detection object is in a prone state when facing downward, the detection object is in a supine state when facing upward, and the detection object is in a decub state when facing one of both sides. Reference may be made to FIG. 2 and FIG. 3 for the supine posture and the decub posture.

FIG. 5 is a schematic diagram of step 402. As shown in FIG. 5, step 402 includes the following steps. Step 501 for determining a posture of the detection object; and step 502 for determining, in response to a determination result of the posture of the detection object, whether an isolation pad is present at the predetermined part of the detection object using at least one of color channel information and depth information of the image.

In some embodiments of operation 501, the posture of the detection object may be obtained based on the color channel information and/or the depth information of the image.

For example, the color image may be input into a predetermined posture determination model to obtain a posture corresponding to the color image. The posture determination model may determine the poster of the detection object based on a classification network, or detect key points of the color image and determine the posture of the object based on a result of detecting the key points (e.g., information such as positions of the key points).

For another example, depth information of key points in the depth image may be compared with depth information templates corresponding to different postures, to determine the posture of the detection object.

In the present application, the key points may be, for example, feature points related to body parts that can be detected in an image. Position information of the key points may be represented by pixel positions in the image. The present application is not limited thereto, and the position information of the key point may be represented in other manners.

The types of the key points may include at least one of the following: head, chest, abdomen, neck, nose, left shoulder, right shoulder, left hip, right hip, left eye, right eye, left elbow, right elbow, left knee, right knee, left ear, right ear, left wrist, right wrist, left ankle, and right ankle. The present application is not limited thereto, and the type of the key point may also include other contents.

For specific details of a method for determining the posture of the detection object in operation 501, reference may be made to the related art. In addition, the method for determining the posture of the detection object may not be limited to the above description, and other methods may also be adopted.

In operation 502, in response to the posture of the detection object being a first posture, whether an isolation pad is present at the predetermined part of the detection object is determined using the color channel information of the image; or, in response to the posture of the detection object being a second posture, whether an isolation pad is present at the predetermined part of the detection object is determined using at least one of the color channel information and the depth information of the image.

In operation 502, the first posture is, for example, a decub posture, etc., and the second posture is, for example, a prone posture or a supine posture, etc. The predetermined part may be, for example, at least one of between the upper limbs, between the upper limb and a side of the trunk, between two legs, and between two feet of the detection object, or may be another part that easily forms a body loop.

In operation 502, determining whether an isolation pad is present at the predetermined part of the detection object using color channel information of the image may be implemented by using a template matching method.

For example, a figure in a predetermined region in the image is resized at least once (i.e., the figure is enlarged or reduced, etc.), and a resized figure is compared with a template (e.g., there is at least one template). In response to a comparison result that the resized figure matches the template, it is determined that an isolation pad is present at the predetermined part of the detection object.

In addition, if the figure is resized many times (e.g., predetermined number N times, N being a natural number greater than or equal to 2) and does not match the template, it is determined that no isolation pad is present at the predetermined part of the detection object.

In at least one embodiment, when the same figure is resized more than twice, an enlargement or reduction ratio of the image is different for each resizing operation. For example, the enlargement or reduction ratio of the image is M1, M2, . . . respectively, for each resizing operation. By means of resizing of the figure, even in a scenario in which a size of a figure in a predetermined region in the image is not fixed due to a height change of a detection bed, the figure can be adjusted to an appropriate size. Therefore, when detection is performed by using the template, an accurate detection result can be obtained, and the occurrence of misdetection or erroneous detection can be avoided.

For example, if the detection bed is low, a distance between the detection object 170 and the camera 180 is large, and the size of the figure in the predetermined region is small; therefore, enlarging the size of the figure by a large multiple can facilitate detection with the template.

For another example, if the detection bed is high, a distance between the detection object 170 and the camera 180 is short, and the size of the figure in the predetermined region is large; therefore, enlarging the size of the figure by a small multiple can facilitate detection with the template.

In at least one embodiment, the template may be used to detect whether there is a predetermined pattern in the figure in the predetermined region. The predetermined pattern may be a pattern arranged on a surface of the isolation pad 201, and the pattern can improve the recognizability of the isolation pad 201, so that the isolation pad can be more accurately detected. For example, the predetermined pattern may be periodically arranged on the surface of the isolation pad 201. In addition, the predetermined pattern may be different from a background of the surface of the isolation pad 201 in at least one aspect of color, shape and texture.

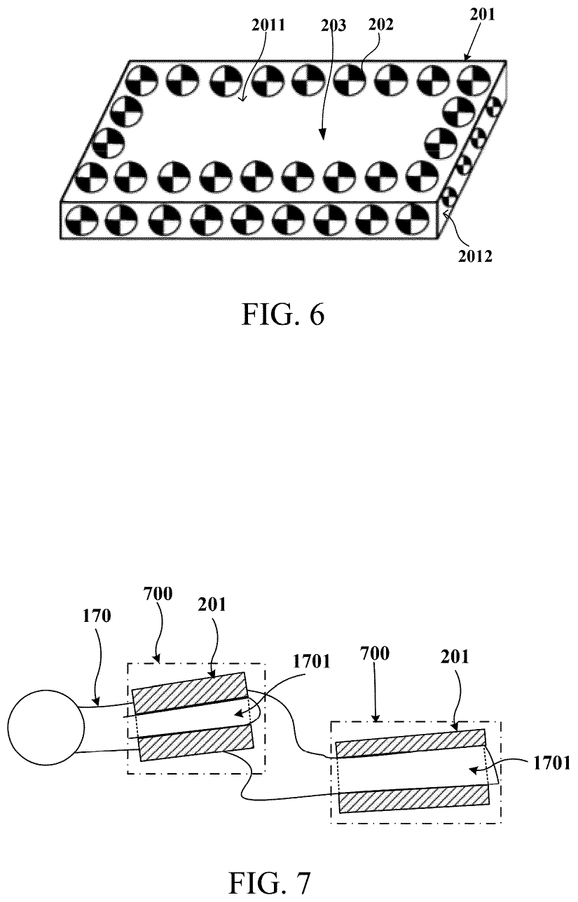

FIG. 6 is a schematic diagram of a predetermined pattern of the isolation pad. As shown in FIG. 6, a pattern 202 is periodically arranged on the surface of the isolation pad 201. At least one of a color, a shape and a texture of the pattern 202 is different from a background 203 of the surface of the isolation pad 201, thereby facilitating recognition of the pattern 202.

As shown in FIG. 6, the pattern 202 may be arranged on a top surface 2011 and a bottom surface (not shown) among surfaces of the isolation pad 201. In addition, the pattern 202 may be arranged in regions of the top surface 2011 and the bottom surface that are not easily obscured by a body part of the detection object, such as an edge part, thereby improving detection efficiency and reducing the cost of arranging the pattern 202.

In addition, the pattern 202 may also be arranged on a side surface 2012 among the surfaces of the isolation pad 201. Therefore, the pattern 202 is arranged on a plurality of surfaces of the isolation pad 201, which can ensure that the pattern 202 can be captured by the camera 180 when the isolation pad 201 and the camera 180 are in different relative positional relationships.

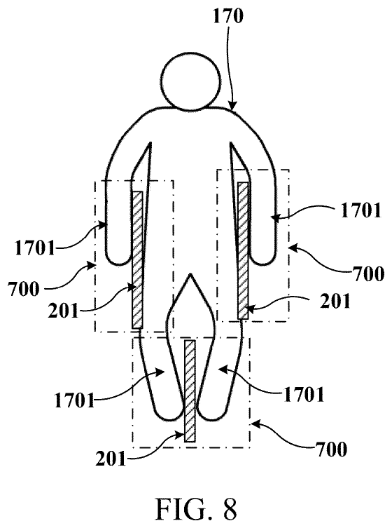

In the present application, the predetermined region involved in operation 502 may refer to a region in the image including the above predetermined part. FIG. 7 is a schematic diagram of an image captured by the camera when the detection object is in the first posture, and FIG. 8 is a schematic diagram of an image captured by the camera when the detection object is in the second posture. As shown in FIG. 7 and FIG. 8, a predetermined region 700 may include a predetermined part 1701 of the detection object 170.

For example, when the predetermined part 1701 of the detection object 170 is detected from the image captured by the camera, the predetermined region 700 may be set in the image based on a position and an area of the predetermined part 1701, so that the predetermined region 700 includes the predetermined part.

In operation 502 of the present application, detection is performed on the predetermined region 700, so that a detection range can be narrowed, thereby increasing a detection speed.

In some examples, the predetermined region 700 may be a rectangular region, but the present application may not be limited thereto, and the predetermined region 700 may also be a region having another shape. In addition, the area of the predetermined region 700 may be set in advance, or may be adjusted based on a detected area of the predetermined part.

In addition, in the present application, the predetermined part 1701 may be detected, from the image captured by the camera using the related art. For example, key points in the image can be detected, and which pixels are pixels of the predetermined part can be determined based on a result of detecting the key points, thereby detecting the predetermined part 1701 in the image.

In operation 502, determining whether an isolation pad is present at the predetermined part of the detection object 170 using the depth information includes: calculating depth difference information in a predetermined region in the image, and, in response to the depth difference information being greater than a predetermined threshold, determining that an isolation pad is present in the predetermined part of the detection object; in addition, if the depth difference information is less than or equal to a predetermined threshold, determining that no isolation pad is present at the predetermined part of the detection object. For the description of the predetermined region, reference may be made to the above description.

The depth difference information may be calculated based on depth information of each pixel in the predetermined region. For example, an absolute value of a difference between a maximum value and a minimum value of depths of pixels in the predetermined region may be calculated, and the absolute value may be used as the depth difference information. For another example, gradient values of depths of pixels in the predetermined region may be calculated, and a maximum value or an average value of absolute values of the gradient values may be calculated as the depth difference information.

In operation 502 of the present application, when the posture of the detection object is the first posture, as shown in FIG. 3, the isolation pad 201 does not protrude from the detection object in a height direction. Therefore, whether an isolation pad is present at the predetermined part of the detection object is determined using the color channel information of the image.

In operation 502 of the present application, when the posture of the detection object is the second posture, as shown in FIG. 2, the isolation pad 201 is clamped by the predetermined part of the detection object, thereby forming a height difference with a surrounding region in a height direction. Therefore, whether an isolation pad is present at the predetermined part of the detection object is determined using the depth information of the image. For example, whether an isolation pad is present at the predetermined part of the detection object is determined using only the depth information of the image; or whether an isolation pad is present at the predetermined part of the detection object is determined using both the depth information and the color channel information of the image. In addition, when the posture of the detection object is the second posture, whether an isolation pad is present at the predetermined part of the detection object can also be determined using only the color channel information of the image.

Determining whether an isolation pad is present at the predetermined part of the detection object using both the depth information and the color channel information of the image may include: determining whether an isolation pad is present at the predetermined part of the detection object using the color channel information of the image to obtain a first determination result; determining whether an isolation pad is present at the predetermined part of the detection object using the depth information of the image to obtain a second determination result; and when both the first determination result and the second determination result for the same predetermined part are that an isolation pad is present at the predetermined part, determining that an isolation pad is present at the predetermined part, and otherwise, determining that no isolation pad is present at the predetermined part.

In at least one embodiment of the present application, information related to a determination result in operation 402 may be output in operation 403. The information may be at least one of text, an image, an icon, a sound, vibration, etc.

For example, if the determination result in operation 402 is that an isolation pad is present at each predetermined part of the detection object, the determination result is output in operation 403. If the determination result in operation 402 is that no isolation pad is present at at least one predetermined part of the detection object, warning information is output in operation 403, to prompt an operator to detect the placement of an isolation pad. In addition, prompt information may also be output in operation 403, the prompt information prompting which predetermined part or parts do not have an isolation pad in the form of images and/or sounds.

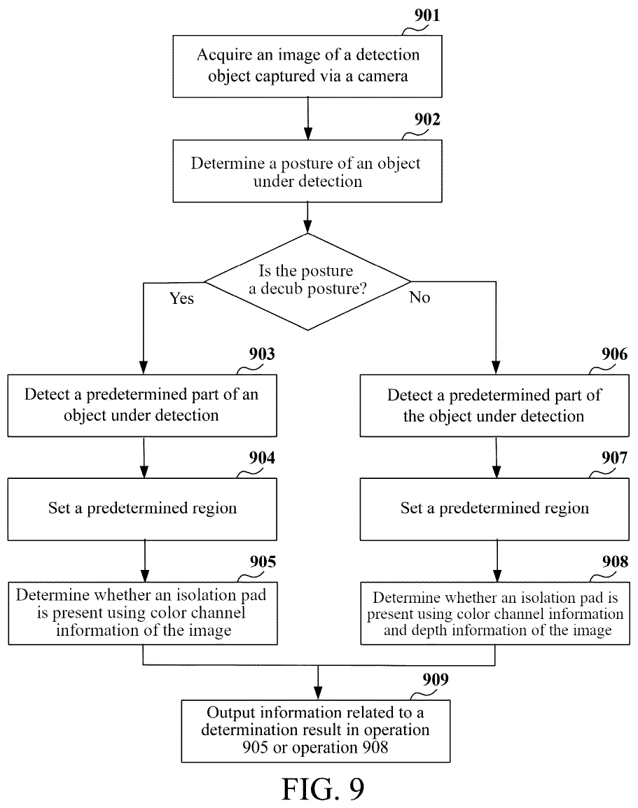

FIG. 9 is a schematic flowchart of a specific implementation of a method for detecting an isolation pad in medical imaging according to an embodiment of the present application. As shown in FIG. 9, the process includes the steps 901-909 as described below.
Step 901: Acquiring an image of a detection object captured via a camera.
Step 902: Determining a posture of the detection object based on the image.
Step 903: Detecting a predetermined part of the detection object when the detection object is in a decub posture, for example, the predetermined part including the elbow and wrist.
Step 904: Setting a predetermined region in the image based on a detection result for the predetermined part in step 903, to surround the predetermined part.
Step 905: Detecting, using color channel information of the image, a predetermined pattern in the predetermined region that is set in step 904, and determining whether an isolation pad is present.
Step 906: Detecting the predetermined part of a detection object (e.g., the predetermined part including the elbow, wrist, knee, and ankle), when the detection object is not in a decub posture (e.g., is in a supine posture or a prone posture).
Step 907: Setting a predetermined region in the image based on a detection result for the predetermined part in step 906, to surround the predetermined part.
Step 908: Determining whether an isolation pad is present at the predetermined part of the detection object using color channel information and depth information of the image.

For example, in step 908: detecting, using the color channel information of the image, a predetermined pattern in the predetermined region that is set in step 907, and determining whether an isolation pad is present, to obtain a first determination result; calculating, using the depth information of the image, depth difference information for the predetermined region that is set in step 907, and determining whether an isolation pad is present based on a result of comparison between the depth difference information and a predetermined threshold, to obtain a second determination result; and when both the first determination result and the second determination result for the same predetermined region are that an isolation pad is present at the predetermined part, determining that an isolation pad is present at the predetermined location, and otherwise, determining that no isolation pad is present at the predetermined part.
Step 909: Outputting information related to a determination result in step 905 or step 908, step 909 being the same as step 403.

According to the above embodiment, an isolation pad placed on the body of the detection object is detected, and therefore, it is possible to reliably avoid the formation of a loop in the body of the detection object.

Further provided in embodiments of the present application is an apparatus for detecting an isolation pad in medical imaging; content the same as that of the foregoing embodiments is not repeated here.

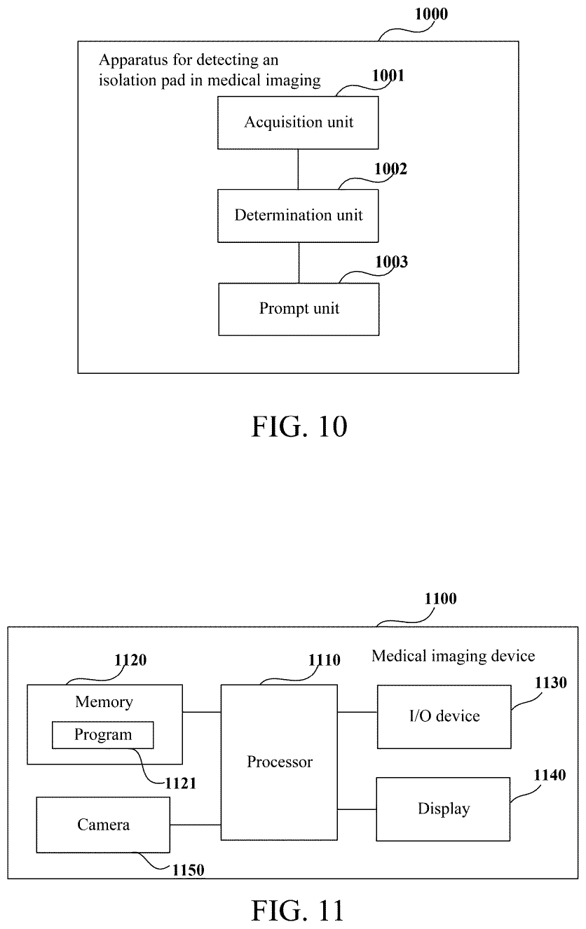

FIG. 10 is a schematic diagram of an apparatus for detecting an isolation pad in medical imaging according to an embodiment of the present application. As shown in FIG. 10, the apparatus 1000 for detecting an isolation pad includes: an acquisition unit 1001 for acquiring an image of a detection object captured via a camera and a determination unit 1002 for determining, based on the image, whether an isolation pad is present at a predetermined part of the detection object. The apparatus 1000 further includes a prompt unit 1003 for outputting information related to a result of the determination.

In some embodiments, the determination unit 1002 determining, based on the image, whether an isolation pad is present at a predetermined part of the detection object includes: determining a posture of the detection object; and determining, in response to a determination result of the posture of the detection object, whether an isolation pad is present at the predetermined part of the detection object, using at least one of color channel information and depth information of the image.

In some embodiments, the determination unit 1002 determines, in response to the posture of the detection object being a first posture, whether an isolation pad is present at the predetermined part of the detection object using color channel information of the image; or

The determination unit 1002 determines, in response to the posture of the detection object being a second posture, whether an isolation pad is present at the predetermined part of the detection object using at least one of color channel information and depth information of the image.

In some embodiments, determining whether an isolation pad is present at the predetermined part of the detection object using the color channel information of the image includes: resizing a figure in a predetermined region in the image at least once, comparing the resized figure with a template, and, in response to a comparison result that the resized figure matches the template, determining that an isolation pad is present at the predetermined part of the detection object.

In some embodiments, the template is used to detect whether there is a predetermined pattern in the figure. For example, the predetermined pattern is periodically arranged on a surface of the isolation pad, and at least one of a color, a shape and a texture of the predetermined pattern is different from a background of the surface of the isolation pad.

In some embodiments, determining whether an isolation pad is present at the predetermined part of the detection object using the depth information includes: calculating depth difference information in a predetermined region in the image, and in response to the depth difference information being greater than a predetermined threshold, determining that an isolation pad is present at the predetermined part of the detection object.

It is worth noting that only the components or modules related to the present application have been described above, but the present application is not limited thereto. The apparatus 1000 for detecting an isolation pad in medical imaging may further include other components or modules, the specifics of which can be found in the related art.

For the sake of simplicity, FIG. 10 only exemplarily illustrates the connection relationships or signal directions between various components or modules, but it should be clear to those skilled in the art that various related technologies such as bus connection can be used. The various components or modules can be implemented by means of hardware such as a processor or a memory, etc. The embodiments of the present application are not limited thereto.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

Embodiments of the present application further provide a medical imaging system. The apparatus 1000 for detecting an isolation pad in medical imaging as described in the embodiment of the second aspect is included, the contents of which are incorporated here. The medical imaging device may, for example, have a computer, a server, a workstation, a laptop computer, a smart phone, or the like. However, the embodiments of the present application are not limited thereto.

FIG. 11 is a schematic diagram of a medical imaging device according to an embodiment of the present application. As shown in FIG. 11, a medical imaging device 1100 may include: one or more processors (for example, central processing units (CPUs)) 1110 and one or more memories 1120. The memory 1120 is coupled to the processor 1110. The memory 1120 may store various types of data. In addition, the memory further stores a program 1121 for information processing, and executes the program 1121 under the control of the processor 1110.

In some embodiments, functions of the apparatus 1000 for detecting an isolation pad in medical imaging are integrated into the processor 1110 for implementation. The processor 1110 is configured to implement the method for detecting an isolation pad in medical imaging as described in the preceding embodiments of the present application.

In some embodiments, the apparatus 1000 for detecting an isolation pad in medical imaging and the processor 1110 are configured separately. For example, the apparatus 1000 for detecting an isolation pad in medical imaging may be configured as a chip connected to the processor 1110, and the functions of the apparatus 1000 for detecting an isolation pad in medical imaging implemented under the control of the processor 1110.

For example, the processor 1110 is configured to perform the following control: acquiring an image of a detection object captured via a camera; determining, based on the image, whether an isolation pad is present at a predetermined part of the detection object; and outputting information related to a result of the determination.

In a specific example, the medical imaging device 1100 of FIG. 11 may be the magnetic resonance imaging (MRI) system 100 shown in FIG. 1. The memory 1120 of FIG. 11 may correspond to at least one of the memory 137 and the memory 126 of FIG. 1. For example, the memory 1120 may be independent of at least one of the memory 137 and the memory 126, or the memory 1120 may be in communication with at least one of the memory 137 and the memory 126, or the memory 1120 may include at least one of the memory 137 and the memory 126, etc. The processor 1110 of FIG. 11 may correspond to at least one of the CPU 131, the CPU 124 and the image processor 128 of FIG. 1. For example, the processor 1110 may be independent of at least one of the CPU 131, the CPU 124 and the image processor 128, or the processor 1110 may be in communication with at least one of the CPU 131, the CPU 124 and the image processor 128, or the processor 1110 may include at least one of the CPU 131, the CPU 124 and the image processor 128, etc.

In addition, as shown in FIG. 11, the medical imaging device 1100 may further include: an input/output (I/O) device 1130, a display 1140, etc. The functions of the foregoing components are similar to those in the prior art. Details are not described herein again.

In addition, as shown in FIG. 11, the medical imaging device 1100 may further include a camera 1150, which captures an object and generates an image. The image may be transmitted to the processor 1110, so that the processor 1110 can implement the method for detecting an isolation pad in medical imaging described in the above embodiments of the present application based on the image captured by the camera 1150.

It is worth noting that the medical imaging device 1100 does not necessarily include all of the components shown in FIG. 11. In addition, the medical imaging device 1100 may further include components not shown in FIG. 11, for which reference may be made to the related art.

Further provided in embodiments of the present application is a computer-readable program, which, when executed in a medical imaging system, causes a computer to perform, in the medical imaging system, the method for detecting an isolation pad in medical imaging described in the foregoing embodiments.

The embodiments of the present application further provide a storage medium having a computer-readable program stored therein, where the computer-readable program causes a computer to execute, in a medical imaging system, the method for detecting the isolation pad in medical imaging according to the foregoing embodiments.

The above apparatus and method of the present application can be implemented by hardware, or can be implemented by hardware in combination with software. The present application relates to the foregoing type of computer-readable program. When executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A method for detecting an isolation pad in medical imaging, wherein the method comprises:
acquiring an image of a detection object captured via a camera;
determining, based on the image, whether an isolation pad is present at a predetermined part of the detection object; and
outputting information related to a result of the determination.

2. The method according to claim 1, wherein
determining, based on the image, whether an isolation pad is present at a predetermined part of the detection object comprises:
determining a posture of the detection object; and
determining, in response to a determination result of the posture of the detection object, whether an isolation pad is present at the predetermined part of the detection object by using at least one of color channel information and depth information of the image.

3. The method according to claim 2, wherein
determining, in response to the posture of the detection object being a first posture, whether an isolation pad is present at the predetermined part of the detection object using color channel information of the image; or
determining, in response to the posture of the detection object being a second posture, whether an isolation pad is present at the predetermined part of the detection object using at least one of the color channel information and depth information of the image.

4. The method according to claim 2, wherein
determining whether an isolation pad is present at the predetermined part of the detection object using color channel information of the image comprises:
resizing a figure in a predetermined region in the image at least once, comparing the resized figure with a template, and, in response to a comparison result that the resized figure matches the template, determining that an isolation pad is present at the predetermined part of the detection object.

5. The method according to claim 4, wherein
the template is used to detect whether there is a predetermined pattern in the figure.

6. The method according to claim 5, wherein
the predetermined pattern is periodically arranged on a surface of the isolation pad, and
at least one of a color, a shape and a texture of the predetermined pattern is different from a background of the surface of the isolation pad.

7. The method according to claim 2, wherein
determining whether an isolation pad is present at the predetermined part of the detection object using the depth information of the image comprises:
calculating depth difference information in a predetermined region in the image, and, in response to the depth difference information being greater than a predetermined threshold, determining that an isolation pad is present at the predetermined part of the detection object.

8. A medical imaging system, comprising a memory and a processor, the memory storing a computer program, and the processor being configured to execute the computer program to implement the method for detecting an isolation pad in medical imaging according to claim 1.

9. An apparatus for detecting an isolation pad in medical imaging, wherein the apparatus comprises:
an acquisition unit, acquiring an image of a detection object captured via a camera;
a determination unit, determining, based on the image, whether an isolation pad is present at a predetermined part of the detection object; and
a prompt unit, outputting information related to a result of the determination.

10. The apparatus according to claim 9, wherein
the determination unit determining, based on the image, whether an isolation pad is present at a predetermined part of the detection object comprises:
determining a posture of the detection object; and
determining, in response to a determination result of the posture of the detection object, whether an isolation pad is present at the predetermined part of the detection object, using at least one of color channel information and depth information of the image.

11. The apparatus according to claim 10, wherein
the determination unit determines, in response to the posture of the detection object being a first posture, whether an isolation pad is present at the predetermined part of the detection object using color channel information of the image; or
the determination unit determines, in response to the posture of the detection object being a second posture, whether an isolation pad is present at the predetermined part of the detection object using at least one of color channel information and depth information of the image.

12. The apparatus according to claim 10, wherein
determining whether an isolation pad is present at the predetermined part of the detection object using color channel information of the image comprises:
resizing a figure in a predetermined region in the image at least once, comparing the resized figure with a template, and, in response to a comparison result that the resized figure matches the template, determining that an isolation pad is present at the predetermined part of the detection object.

13. The apparatus according to claim 12, wherein
the template is used to detect whether there is a predetermined pattern in the figure.

14. The apparatus according to claim 13, wherein
the predetermined pattern is periodically arranged on a surface of the isolation pad, and
at least one of a color, a shape and a texture of the predetermined pattern is different from a background of the surface of the isolation pad.

15. The apparatus according to claim 10, wherein
determining whether an isolation pad is present at the predetermined part of the detection object using the depth information of the image comprises:
calculating depth difference information in a predetermined region in the image, and in response to the depth difference information being greater than a predetermined threshold, determining that an isolation pad is present at the predetermined part of the detection object.
